(19) United States
(12) Patent Application Publication
(10) Pub. No.: US 2022/0073964 A1
(43) Pub. Date: Mar. 10, 2022

(54) NUCLEIC ACID DETECTION

(71) Applicant: Hewlett-Packard Development Company, L.P, Spring, TX (US)

(72) Inventor: George H. Corrigan, III, Corvallis, OR (US)

(73) Assignee: Hewlett-Packard Development Company, L.P., Spring, TX (US)

(21) Appl. No.: 17/417,364

(22) PCT Filed: Mar. 7, 2019
(86) PCT No.: PCT/US2019/021148

§ 371 Date: Jun. 22, 2021

(51) Int. Cl. C12Q 1/686; B01L 7/00; B01L 3/00; C12Q 1/6806; G01N 27/327

(52) U.S. Cl. CPC C12Q 1/686; B01L 7/52; B01L 3/502715; B01L 2300/0663; G01N 27/3277; B01L 2300/0645; C12Q 1/6806

(57) ABSTRACT

The present disclosure relates to nucleic acid detection devices. The nucleic detection device can include a microfluidic architecture to receive biological fluid, a multimodal sensor bundle, a common transmission line to transmit an enhanced current signal generated by combining current signals from individual sensors, and a current to voltage converter to receive and convert the enhanced current signal to voltage. The multimodal sensor bundle can be positioned to interact with the biological fluid when present within the microfluidic architecture. The multimodal sensor bundle can include multiple types of sensors selected from an electrochemical sensor, an optical sensor, or a potentiometric sensor. Individual sensors of the multimodal sensor bundle independently can generate a current signal in response to interaction with the biological fluid.

### BACKGROUND

Nucleic acid amplification is a technique utilized in research, medical diagnostics, and forensic testing. The ability to amplify a small quantity of a sample of a nucleic acid to generate copies of the nucleic acid in the sample can permit research, medical diagnostic, and forensic tests that would not otherwise be permissible from the small quantity of the sample, for example.

### BRIEF DESCRIPTION OF THE DRAWINGS

FIG. 1 graphically illustrates a schematic view of an example nucleic acid detection device in accordance with examples of the present disclosure;

FIG. 2 graphically illustrates a schematic view of an example nucleic acid detection device with multiple multimodal sensor bundles and sensor masks in accordance with examples of the present disclosure;

FIG. 3 graphically illustrates a schematic view of an example nucleic acid detection arrayed system in accordance with examples of the present disclosure;

FIG. 4 graphically illustrates a schematic view of an example nucleic acid detection arrayed system with sensor masks in accordance with examples of the present disclosure;

FIG. 5 graphically illustrates a schematic view of an example nucleic acid detection arrayed system with sensor masks and multiple current generating sensors individually present with an individual type of sensor in accordance with examples of the present disclosure;

FIG. 6 graphically illustrates a schematic view of an example nucleic acid detection arrayed system associated with a thermal cycler in accordance with examples of the present disclosure;

FIG. 7 graphically illustrates a schematic view of an example nucleic acid detection arrayed system in accordance with an example of the present disclosure;

FIG. 8 is a flow diagram illustrating an example method of nucleic acid detection in accordance with examples of the present disclosure; and

FIG. 9 graphically illustrates a schematic view of sensor outputs generated from one to five optical sensors using a device similar to that shown FIG. 7.

### DETAILED DESCRIPTION

Nucleic acid amplification can include denaturing, annealing, and extending nucleic acid chains. During denaturing, an increased temperature can cause hydrogen bonds between bases in a double-stranded nucleic acid sample to break apart, resulting in two single strands realized from a formerly double-stranded nucleic acid. During annealing, the heated sample can then be cooled, enabling single stranded nucleic acid oligomers, such as primers, to attach to the complimentary nitrogen bases on the single strands of the nucleic acid. During extending of the nucleic acid chain, the temperature may be increased, for example, to enable a polymerase enzyme to extend the nucleic acid strand by adding nucleic acid bases. In real-time nucleic acid amplification, the amplification of a nucleic acid sample is monitored during the amplification. This can permit the quantification of nucleic acid concentration, gene expression, and sequencing. Detecting nucleic acids can be difficult when nucleic acids are present at a low concentration. In accordance with examples of the present disclosure, a device that combines the output from multiple sensors of the same type and can be programmatically scaled for increased or decreased sensitivity can permit the detection of nucleic acids at low concentrations, can reduce signal-to-noise disturbances, and can permit a wide dynamic sensing range.

In accordance with an example of the present disclosure, a nucleic acid detection device includes a microfluidic architecture to receive biological fluid, a multimodal sensor bundle, a common transmission line to transmit an enhanced current signal generated by combining current signals from individual sensors, and a current to voltage converter to receive and convert the enhanced current signal to voltage. The multimodal sensor bundle in this example is positioned to interact with the biological fluid when present within the microfluidic architecture. The multimodal sensor bundle includes multiple types of sensors selected from an electrochemical sensor, an optical sensor, or a potentiometric sensor. Individual sensors of the multimodal sensor bundle independently generate a current signal in response to interaction with the biological fluid. In one example, the nucleic acid detection device includes a sensor mask associated with the multimodal sensor bundle. The sensor mask is electrically associated with the multimodal sensor bundle to enable or disable the individual sensors of the multimodal sensor bundle. The nucleic acid detection device also includes multiple multimodal sensor bundles independently associated with a common transmission line to transmit an enhanced current signal generated by combining current signals from the individual sensors within the multiple multimodal sensor bundles, respectively. The multimodal sensor bundle (or bundles), in another example, includes the electrochemical sensor, the optical sensor, and the potentiometric sensor. The electrochemical sensor includes a working electrode, a counter electrode, and a reference electrode. The optical sensor includes a photo-diode and a thin film filter. The nucleic acid detection device also includes a thermal cycler associated with the microfluidic architecture to thermally cycle the biological fluid when present within the microfluidic architecture.

In another example, a nucleic acid detection arrayed system includes a microfluidic architecture to receive biological fluid, an array of multimodal sensor bundles positioned to interact with the biological fluid when present within the microfluidic architecture, a series of common transmission lines to transmit an enhanced current signal generated by combining current signals from multiple individual sensors across the array, and a current to voltage converter to receive and convert the enhanced current signal to voltage. Individual multimodal sensor bundles of the array in this example include multiple types of sensors selected from an electrochemical sensor, an optical sensor, or a potentiometric sensor. Individual sensors of the multimodal sensor bundles independently generate current signal in response to interaction with the biological fluid. In one example, the array of multimodal sensor bundles includes the optical sensor in the form of a multi-channel optical sensor with a red channel, a green channel, and a blue channel. Current signal generated by the red channel of multiple optical sensors across the array transmit in parallel to a first common transmission line, current signal generated by the green channel of multiple optical sensors across the array transmit in parallel to a second common transmission line, and current signal generated by the blue channel of multiple optical sensors across the array transmit in parallel to a third common transmission line. In another example, the multimodal sensor bundles include the potentiometric sensor and one or both of the electrochemical sensor or the optical sensor. In yet another example, the series of common transmission lines, on the other hand, are independently associated with a series of transmission gates, respectively, to independently gate the common transition lines prior to introduction of the enhanced current signal to the current to voltage converter. In a further example, the current to voltage converter includes an amplifier. In one example, the microfluidic architecture includes microfluidic microchannels, microfluidic chambers, or both. The microfluidic architecture further includes a microfluidic fluid movement network to generate fluid movement within the microfluidic architecture. In still further detail, the biological fluid is present within the microfluidic architecture and includes a nucleic acid amplifying solution.

In another example, a method of nucleic acid detection includes loading a biological fluid including nucleic acid amplifying components into a microfluidic architecture of a nucleic acid detection device or nucleic acid detection arrayed system, which includes a multimodal sensor bundle that individually includes multiple types of sensors selected from an electrochemical sensor, an optical sensor, or a potentiometric sensor. The method further includes amplifying a nucleic acid within the microfluidic architecture using the nucleic acid amplifying fluid to generate an amplified biological fluid, generating multiple current signals from the multimodal sensor bundle in response to interaction between the amplified biological fluid and multiple sensors including sensors of different types, combining current signals from the multiple sensors of the same type, of a different type, or both to generate an enhanced current signal, and converting the enhanced current signal to voltage. The device or arrayed system is as described above, for example. In one example, the method includes digitally enabling or disabling electrochemical sensors, optical sensors, potentiometric sensors, or a combination thereof using a sensor mask associated with the microfluidic architecture. In another example, the method includes gating the enhanced current signal to provide unidirectional current flow prior to converting the enhanced current signal to voltage.

When discussing the nucleic acid detection device, the nucleic acid detection arrayed system, or the method of nucleic acid detection herein, such discussions can be considered applicable to one another whether or not they are explicitly discussed in the context of that example. Thus, for example, when discussing an electrochemical sensor in the context of a nucleic acid detection device, such disclosure is also relevant to and directly supported in the context of the nucleic acid detection arrayed system and/or the method of nucleic acid detection, and vice versa.

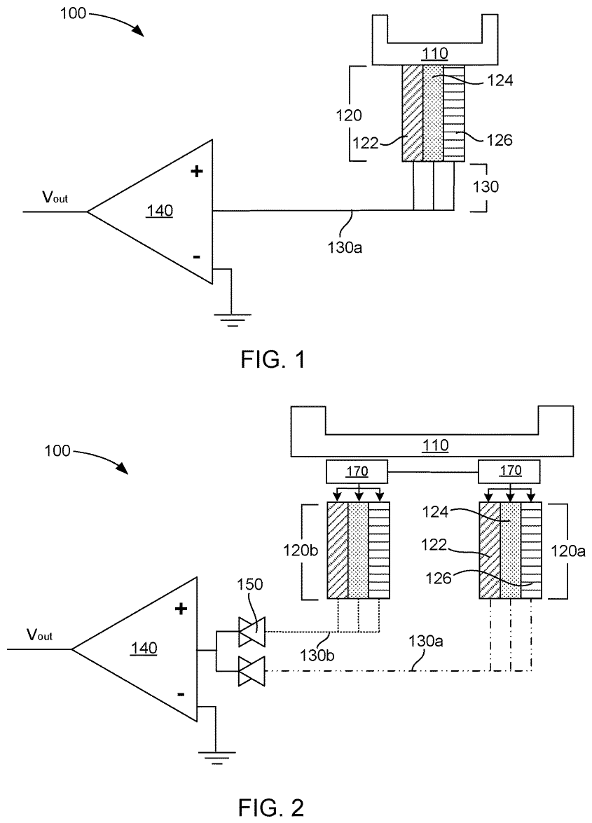

The term “nucleic acid detection device” herein refers to microfluidic apparatuses that include a multimodal sensor bundle positioned to interact with the biological fluid of the microfluidics, and which can generate current signal to be combined along common transmission line(s). There can be one or multiple common transmission lines that can combine signal from different types of sensors within a common multimodal sensor bundle. This can be repeated using multiple multimodal sensor bundles independently associated with a common transmission line to transmit an enhanced current signal generated by combining current signals from the individual sensors within the multiple multimodal sensor bundles, respectively. Examples are respectively shown in FIGS. 1 and 2 of a nucleic acid detection device. That stated, there may or may not be some transmission lines that do not combine signal. The nucleic detection device can also include a current to voltage converter of some type to convert combined current signal and/or uncombined current signal in some examples to voltage, for example.

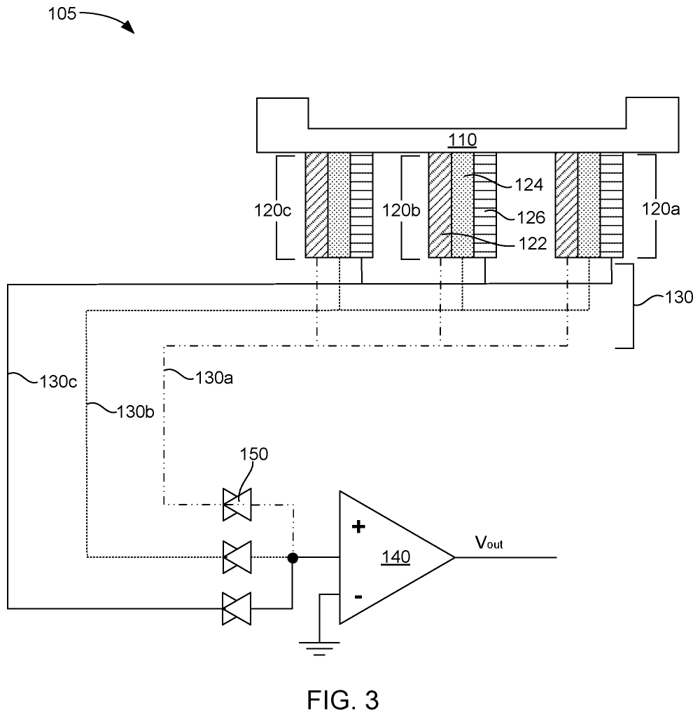

The term “nucleic acid detection arrayed system” refers to microfluidic apparatuses that include an array of multimodal sensor bundles. Thus, though a nucleic acid detection device can include a single multimodal sensor bundle, or an array of multimodal sensor bundles, the nucleic acid detection arrayed system includes multiple multimodal sensor bundles. In this arrangement as an arrayed system, there can be additional flexibility regarding the combining of current from the array of multimodal sensor bundles. For example, in the arrayed system arrangement, like sensors across the array can generate a current in parallel that is then combined to a common transmission line. Other combinations of parallel current lines can be across the array, can be within an individual multimodal sensor bundle, or can be combined into multiple common transmission lines, depending on design parameters. Examples are shown in FIGS. 3 to 6 of various nucleic acid detection arrayed systems. That stated, there may or may not be some transmission lines that do not combine signal. The nucleic detection arrayed system can also include a current to voltage converter of some type to convert combined current signal and/or uncombined current signal in some examples to voltage, for example.

In accordance with the definitions and examples herein, FIGS. 1 and 2 depict various nucleic acid detection devices at 100, and FIGS. 3 to 6 depict various nucleic acid detection arrayed systems at 105. These various examples can include various features, with several features common from example to example. Thus, the reference numerals used for FIGS. 1 to 6 are the same throughout to avoid redundancy, even though the nucleic acid detection device and the nucleic acid detection arrayed system can be electrically wired slightly differently, and the various individual sensors can combine current signal differently, as shown.

In FIGS. 1 and 2, with initial emphasis on the example shown in FIG. 1, a nucleic acid detection device 100 can include a microfluidic architecture 110 to receive biological fluid. A multimodal sensor bundle 120 can include multiple types, e.g., two, three, two plus other type(s) of sensor(s), three plus other type(s) of sensors, etc., of sensors selected from an electrochemical sensor 122, an optical sensor 124, or a potentiometric sensor 126. Parallel transmission lines 130 can be used to combine current, which can then be converted to voltage using a voltage converter 140. Thus, the current generated from the various types of sensors can be transmitted along a common transmission line.

FIG. 2 can be a similar nucleic acid detection device 100 as that shown in FIG. 1, but in this example, there are multiple multimodal sensor bundles 120a, 120b associated with multiple common transmission lines 130a, 130b, respectively. Transmission gates 150 can be positioned along the respective common transmission lines. The transmission gates can act as a relay thereby blocking or permitting a current on a common transmission line from entering the current to voltage converter. In some examples, another type of switch can be used in place of a transmission gate (not shown) in this or any of the other examples herein. Other types of gates or switches or assemblies can likewise be used. The ability to turn on or off the signal that enters the current to voltage converter can allow for selective measurement of a signal generated by a specific group of sensors of a multimodal sensor bundle. Also in this example (or in the example of FIG. 1), there can be sensor masks 170 that can be individually associated with specific multimodal sensor bundles. The sensor masks can be implemented with a serial shifter and can be zone based to enable or disable an interaction between an individual sensor and a biological fluid, when present, in the microfluidic architecture 110. For example, a sensor mask can be used to block an interaction between an optical sensor and a biological fluid while permitting an interaction between an electrochemical sensor and a biological fluid and/or permitting an interaction between a potentiometric sensor and a biological fluid and vice versa. In some examples, the sensor masks can be provided with a mask bit, a common enable signal, or a combination thereof. The electrochemical sensors 122, optical sensors 124, and/or potentiometric sensors 126 can be similar to that shown in FIG. 1, but they can be present in multiple multimodal sensor bundles.

Turning now more specifically to the nucleic acid detection arrayed systems 105 shown in FIGS. 3 to 6, as mentioned, many of the components can be the same or similar to those described with respect to the nucleic acid detection devices. However, with these arrayed systems, for example, a common transmission line can individually receive current signal from sensors across the array to be combined. For example, as shown in FIG. 3, multiple electrochemical sensors 122 across an array of multimodal sensor bundles 120a, 120b, 120c feed parallel transmission lines 130, which in turn feeds common transition line 130a. Likewise, multiple optical sensors 124 across the array of multimodal sensor bundles feed electrical current in parallel to common transition line 130b. Furthermore, multiple potentiometric sensors 126 across the array of multimodal sensor bundles feed electrical current in parallel to common transition line 130c. Similar to that shown in FIG. 2, there can also be a microfluidic architecture 110 to carry fluid to areas for sensing to occur, a voltage converter 140 to convert current to voltage, and transmission gates 150 to act as a relay for blocking or permitting a current on a common transmission line from entering the current to voltage converter. The ability to turn on or off the signal that enters the current to voltage converter can allow for selective measurement of a signal generated by sensors of the multimodal sensor bundles.

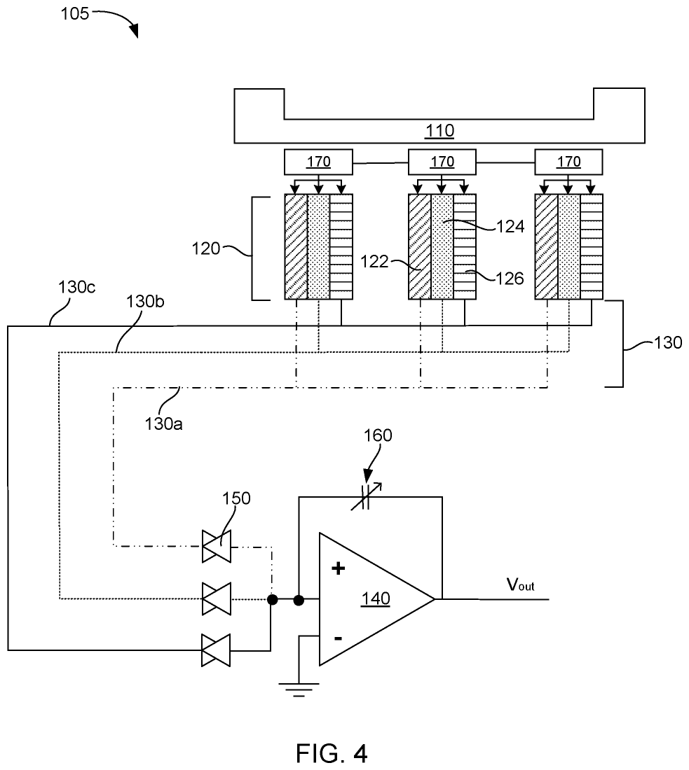

In FIG. 4, the nucleic acid detection arrayed system 105 can be the same or similar to that shown in FIG. 3 (with the same reference numerals and description applicable), but in this example, the arrayed system can further include a sensor mask 170 and/or feedback circuitry 160. The sensor mask can include, for example, a register or register array, e.g., serial shiftable or addressable register. For example, sensor masks can be individually associated with individual multimodal sensor bundles, and the sensor masks can be implemented with a serial shifter to provide for zone-based shifting to enable or disable an interaction between an individual sensor and a biological fluid. Other types of on/off mechanisms can be used to selectively operate the sensor mask, e.g., mechanical movement, electrical modification, optical filtering, etc. To illustrate further, a sensor mask can be used to block an interaction between an optical sensor and a biological fluid while permitting an interaction between an electrochemical sensor and a biological fluid and/or permitting an interaction between a potentiometric sensor and a biological fluid. Other combinations of on/off functionality can be implemented. In still further detail, sensor masks can be provided with a mask bit, a common enable signal, or a combination thereof. In this FIG., the individual multimodal sensor bundles are not shown as 120a, 120b, and 120c, but notably there are three multimodal sensor bundles present in this nucleic acid detection arrayed system 105. There could be fewer, e.g., 0.2, or more, e.g., 4 to 4,096 or an n×m row column array of 1,024×1,024=1,048,576 bundles.

With respect to the feedback circuitry, current traveling on common transmission lines (after selective gating in this example) can be transmitted to a current to voltage converter 140. In this example, and other examples herein the voltage converter can include a transimpedance amplifier, a charge amplifier, and/or a resistor, for example. The feedback circuitry can, for example, be capacitive or resistive, and in some examples, can be adjustable. The adjustment can be controlled by control bits, transmission gates, reset switches, or a combination thereof. In another example, the current to voltage converter can further include an amplifier. The voltage exiting the current to voltage converter can reflect a summation of the signals generated from multiple sensors in the multimodal sensor bundles.

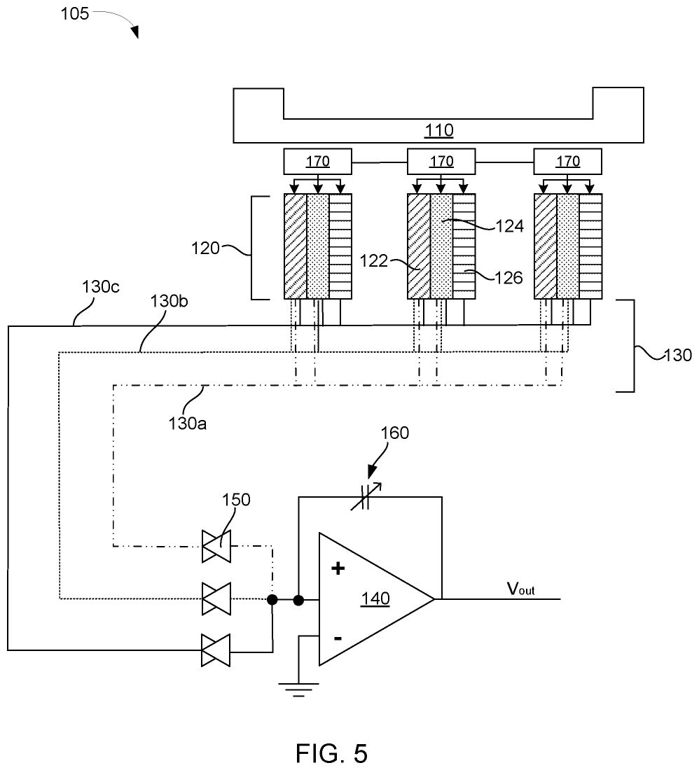

In FIG. 5, the nucleic acid detection arrayed system 105 can be the same or similar to that shown in FIG. 4 (with the same reference numerals and description applicable), but in this example, the arrayed system includes parallel transmission lines 130 from individual sensors, e.g., three from individual electrochemical sensors 122, three from the individual optical sensors 124, and one from the individual potentiometric sensors 126. These parallel lines are shown by way of example, and can be another number of lines from an individual sensor.

By way of specific example, an electrochemical sensor 122 from an individual multimodal sensor bundle 120 can include a working electrode, a counter electrode, and a reference electrode as the three parallel transmission lines that feed three separate common transmission lines, respectively. In this FIG. the individual multimodal sensor bundles are not shown as 120a, 120b, and 120c, but notably there are three multimodal sensor bundles present in this nucleic acid detection arrayed system 105. There could be fewer, e.g., 0.2, or more, e.g., 4 to 4,096. Thus, current signal generated by the working electrode of multiple electrochemical sensors across the array of multimodal sensor bundles can feed common transmission line 130a, current signal generated by the counter electrode of multiple electrochemical sensors across the array of multimodal sensor bundles can feed common transmission line 130b, and current signal generated by the reference electrode of multiple electrochemical sensors across the array of multimodal sensor bundles can feed common transmission line 130c These choices can be arbitrary, or can be strategic based on when certain signals that use a common transmission line may be used in sequence, for example.

By way of another specific example, an optical sensor 124 from an individual multimodal sensor bundle 120 can include a red channel, a green channel, and a blue channel. Thus, current signal generated by the red channel of multiple optical sensors across the array of multimodal sensor bundles can feed common transmission line 130a, current signal generated by the green electrode of multiple optical sensors across the array of multimodal sensor bundles can feed common transmission line 130b, and current signal generated by the blue channel of multiple optical sensors across the array of multimodal sensor bundles can feed common transmission line 130c. Again, these choices can be arbitrary, or can be strategic based on when certain signals that use a common transmission line may be used in sequence, for example.

In other examples, a series of common transmission lines can be combined with an array of multimodal sensor bundles where sensors in individual multimodal sensor bundles include multiple sensing modalities. In this example, individual common transmission lines can combine and transmit multiple signals generated from different sensing modalities across multiple sensor types along common transmission lines. For example, in a multimodal sensor bundle including an electrochemical sensor with a working electrode, a counter electrode, and a reference electrode can combine current with a multi-channel optical sensor with a red channel, a green channel, and a blue channel. Thus, by way of example, current generated from the working electrode and the red channel can be transmitted along transmission line 130a at the same time or at different times (alternating with any timing, pulse, pattern, etc.). Current generated from the counter electrode and the green channel can be transmitted along transmission line 130b at the same time or at different times. Current generated from the reference electrode and the blue channel can be transmitted along a transmission line 130c at the same time or at different times. In this example, the current generated from a potentiometric sensor can be transmitted along any of the three common transmission lines, either at the same time or alternating with one or multiple current signals. In FIG. 5, the potentiometric sensor is established along common transmission line 130c by example.

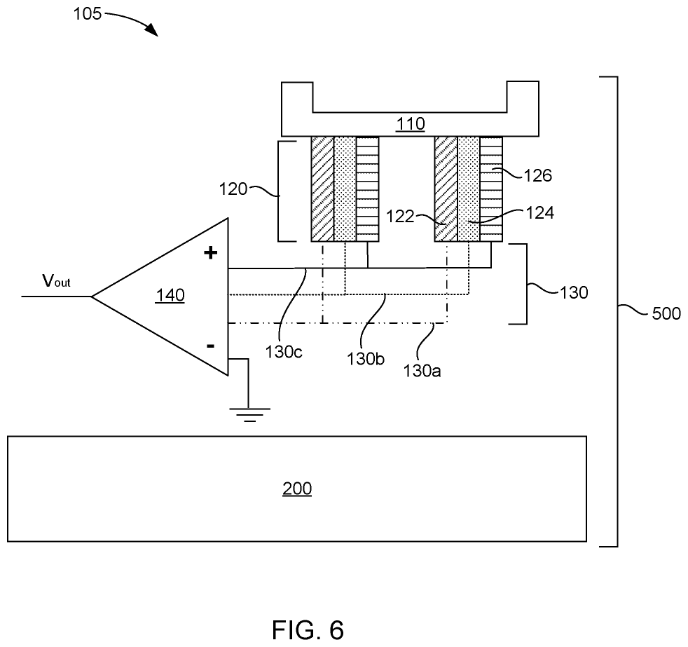

In another example, as shown in FIG. 6, a nucleic acid detection arrayed system 105 (which can likewise be a nucleic acid detection device such as that shown in FIGS. 1 and 2) can include similar features as those described previously, with the same reference numerals thereof. However, in this example, the arrayed system (or device as applicable) can further include a thermal cycler 200. The thermal cycler can be an internal thermal cycler or can be an external thermal cycler. Thus, in this example, the nucleic acid detection arrayed system can include a microfluidic architecture 110 to receive biological fluid; multimodal sensor bundles 120, which individually include multiple types of sensors selected from an electrochemical sensor 122, an optical sensor 124, or a potentiometric sensor 126; parallel transmission lines 130 which can include common transmission lines 130a, 130b, 130c; and a current to voltage converter 140. No sensor masks, transmission gates, or feedback circuitry, are shown in this example, but they could be implemented as previously described. In still further detail, the arrayed system and/or device can further include a biological fluid within the microfluidic architecture, or can include a biological fluid to be loaded in the microfluidic architecture. The biological fluid can be, for example, a nucleic acid amplifying solution. An example of such a nucleic acid amplifying solution may include a nucleic acid oligomer, and a redox-active intercalating dye and/or a fluorescent or fluorescing intercalating dye. In this FIG., the individual multimodal sensor bundles are not shown as 120a, 120b, but notably there are two multimodal sensor bundles present in this nucleic acid detection arrayed system 105. There could be more, e.g., 3 to 4,096.

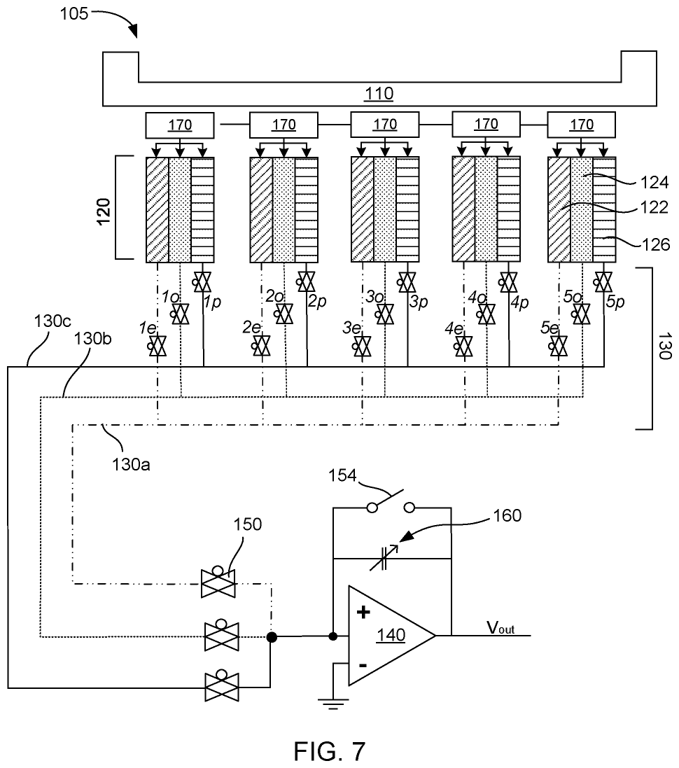

In another example, as shown in FIG. 7, a nucleic acid detection arrayed system 105 can include multiple electrochemical sensors 122 across an array of multimodal sensor bundles 120 can feed parallel transmission lines 130, which in turn feeds common transition line 130a. Likewise, multiple optical sensors 124 across the array of multimodal sensor bundles feed electrical current in parallel to common transition line 130b. Furthermore, multiple potentiometric sensors 126 across the array of multimodal sensor bundles feed electrical current in parallel to common transition line 130c. In this particular example, the parallel transmission lines from the various individual sensor can independently include sensor transmission gates 152, labeled as first electrochemical sensor transmission gate (1e), first optical sensor transmission gate (1o), first potentiometric sensor transmission gate (1p), second electrochemical sensor transmission gate (2e), second optical sensor transmission gate (2o), and so forth. Independent sensor transmission gates could also be included for multiple parallel current lines that may be transmitted from a single sensor, such as that shown in FIG. 5, e.g. one electrochemical sensor could include three independent sensor transmission gates, one optical sensor could include three independent sensor transmission gates, etc. Thus, the ability to turn on or off the signal that is transmitted to the common transmission line(s), or to turn on or off the combined signal that enters the current to voltage converter can allow for selective measurement of a signal generated by sensors of the multimodal sensor bundles. The latter, as shown in prior examples, can be carried out using transmission gates 150 associated with common transmission line(s) to act as a relay for blocking or permitting a current on a common transmission line from entering the current to voltage converter. In this example, and in prior example FIGS., the transmission gates can be used at various locations, depending on the design of the device or system. Transmission gates, simple switches, or other similar switching electrical circuits can be controlled by a digital control circuit that configures the operating parameters of the circuit. In further detail with respect to this example, there may also be a microfluidic architecture 110 to carry fluid to areas where sensing may occur relative to individual sensor bundles. A voltage converter 140, which can include an amplifier with a reset switch 154 to convert current to voltage can be included, can be used. Notably, the reset switch is shown in this example, but the amplifiers/voltage converters of the prior examples may also include a reset switch. Charge-based amplifier typically included reset switches so this feature is not explicitly shown in previous FIGS., but can be present. Furthermore, as with FIG. 4, the nucleic acid detection arrayed system can likewise include a sensor mask 170 and/or feedback circuitry 160 that operates as previously described.

In further detail regarding the microfluidic architecture 110 shown in FIGS. 1-7, this structure can be configured for receiving, moving, mixing, depositing, etc., biological fluids as may be applicable for a specific assay. The microfluidic architecture can include microfluidic microchannels, microfluidic chambers, or a combination thereof and can be located with respect to a multimodal sensor bundle to permit sensing of a biological fluid when present in the microfluidic architecture by sensors of a multimodal sensor bundle. In some examples, the microfluidic architecture can include a single microfluidic chamber associated with an array of multimodal sensor bundles that can be located under the chamber or can include multiple microfluidic chambers individually associated with a multimodal sensor bundle. In some examples, the microfluidic architecture can further include a microfluidic fluid movement network to generate fluid movement within the microfluidic architecture. The microfluidic fluid movement network can include multiple pumps to generate fluid flow through the microfluidic architecture. The microfluidic fluid movement network, for example, can include any combination of pumps that can generate fluid flow such as an inlet pump, an outlet pump, an inertial pump, a fluid ejector, a drop ejector, a DC electroosmotic pump, an AC electroosmotic pump, a diaphragm pump, a peristaltic pump, a capillary pump, or a combination thereof. A fluid or drop ejector can include pumps that can operate in the same way as piezo inkjet printheads or thermal inkjet printheads, ejecting fluid from one microfluidic channel in a direction away from the channel and into a chamber, into another microfluidic channel, or to the environment outside of the nucleic acid amplification device.

As mentioned, the multimodal sensor bundles can include multiple types of sensors. In some examples, individual types of sensor can detect the same nucleic acid target. In other examples, different types of sensors can detect a different nucleic acid target. The types of sensors can be selected from an electrochemical sensor, an optical sensor, a potentiometric sensor, or any combination thereof. Other types of sensors can likewise be used with a plurality of these types of sensors. For example, the multimodal sensor bundle can include an electrochemical sensor and an optical sensor. In another example, the multimodal sensor bundle can include an electrochemical sensor and a potentiometric sensor. In yet another example, multimodal sensor bundle can include an optical sensor and a potentiometric sensor. In a further example, a multimodal sensor bundle can include an electrochemical sensor, an optical sensor, and a potentiometric sensor.

An electrochemical sensor can include an electrode enclosed in a housing. In some examples, an electrochemical sensor can include multiple electrodes. For example, an electrochemical sensor can include a working electrode, a counter electrode, and a reference electrode. The working electrode can be structurally configured to be capable of developing an electrical signal in response to a redox-active compound, e.g., intercalating with a double-stranded nucleic acid or by some other chemically attractive or attaching mechanism. The reference electrode can be constructed to have a stable and characterized electrode potential, and can be used in a half-cell, or in some other cell configurations, and can be used to provide a reference for evaluating a biological fluid based on its electrochemical signature. In some examples, the individual electrochemical sensors can include a reference electrode. In other examples the reference electrode can be a common electrode for an array of electrochemical sensors. The counter-electrode can close an electric circuit and balance a reaction occurring at the working redox electrode. In one example, a counter-electrode can act electrically in concert with respect to the working electrode to thereby provide a circuit over which a current can be measured and evaluated for an electrical signature with respect to the reference electrode. In some examples, the counter-electrode can be a common counter-electrode (a shared counter electrode) for multiple working electrodes and may not be present in individual electrochemical sensors of a multimodal sensor bundles.

An optical sensor can convert a fluorescence emission into an electronic signal, e.g. a current. In one example, the optical sensor can include a multi-channel optical sensor, a pin-photodiode, an avalanche photodiode, a phototransistor, a multi-junction photodiode, a charge coupling device, a complimentary metal-oxide semiconductor, a photo-sensor, a photo-resistor, or a combination thereof. In one example, the optical sensor can be a photo-diode. In another example, the optical sensor can be a multi-channel optical sensor. Multi-channel optical sensors can measure a fluorescence emission at various wavelengths. In one example, a multi-channel optical sensor can include a red channel, a green channel, and a blue channel.

In some examples, an optical sensor can be combined with a filter. The filter can be used to selectively pass or selectively eliminate wavelengths before a wavelength reaches the optical sensor. In some examples the filter can be a thin film filter, a dye filter, or a dichroic filter. In one example, the optical sensor can include a photo-diode and a thin film filter.

A potentiometric sensor can include a voltage divider for measuring changes in electric potential. In one example, the potentiometric sensor can include an ion-selective electrode transducer, chemFET, BioFET, ISFET, or ENFET ion-sensitive field effect transistor structure. The potentiometric sensor can convert a chemical reaction potential directly into a current signal that can be processed or combined with other current signals in the system.

The quantity of bundles can be present to the extent that the physical size the nucleic acid detection device or the nucleic acid detection arrayed system permits. In one example, the quantity of multimodal sensor bundles can range from 1 to any number of bundles in a linear or an n×m array. For example, an n×m row column array can have 1,024×1,024=1,048,576 multimodal sensor bundles or can have 256×256=65,536 multimodal sensor bundles. In some examples, the quantity of multimodal sensor bundles can range from 1 to 1,500,000, from 250 to 1,250,000, from 5,000 to 500,000, from 1,000 to 100,000, from 1 to 150, 2 to 16, from 5 to 20, from 25 to 75, from 100 to 500, etc. In another example, an array of multimodal sensor bundles can be arranged with row-column memory type architecture.

In some examples, the multimodal sensor bundles can have sensing zones that be identically sized. In other examples, the multimodal sensor bundles can have sensing zones of different sizes. Differently sized sensor zones can be scaled by a factor, such as a log factor, to permit summation.

The sensors in the multimodal sensor bundle can independently generate a current in response to interaction with a biological fluid. A sensor that can include multiple sensing modalities (e.g. an electrochemical sensor with a working, reference, and counter electrode or an optical sensor with multiple channels) can generate multiple currents; one for the various individual sensing modalities.

The components discussed above, can be positioned on a substrate. In some examples, the substrate can include a silicon substrate, a silicon germanium substrate, a gallium arsenic substrate, a glass substrate, a plastic, a ceramic, a composite, a metal oxide, a printed circuit board, or a combination thereof. In one example, the substrate can be a silicon substrate.

The nucleic acid detection device or the nucleic acid detection arrayed system can detect current measurements ranging from 0.01 nA to 1 A. In yet other examples the nucleic acid detection device can detect current measurements ranging from 0.01 nA to 1 μA, from 0.01 μA to 10 mA, from 1 μA to 100 mA, or from 100 μA to 1 A.

The nucleic acid detection device described herein can permit signals to be easily summed with precision and can be digitally scalable. In one example, the summation can occur from multiple sensors of the same type, with no significant loss of accuracy. As used herein, “no significant loss” of accuracy means that the summation does not result in more than 5% deviation of measured result. In some examples, the summation does not result in more than 1% deviation of measured result. In yet other examples, the summation can occur from multiple sensors of different types. The ability to sum signals can permit scaling for increased or decreased sensitivity and detection using different fluid volumes. The scaling ability can further allow for diverse detection and can minimize signal-to-noise interference.

Regardless of the configuration of the nucleic acid detection device or the nucleic acid detection arrayed system, in some examples, the apparatus can be integrated on a microfluidic chip, such as a lab-on-a-chip device. In one example, the microfluidic chip can be an integrated point of care diagnostic device, such as an in vitro diagnostic point of care device.

Applications can include nucleic acid amplification, where concentrated nucleic acids may be moved into a microfluidic chamber(s) for amplification and detection while carrying out thermal cycling. Amplification processing can include strand displacement assays, transcription mediated assays, isothermal amplifications, loop mediated isothermal amplification, reverse-transcription loop mediated isothermal amplification, nucleic acid sequence based amplification, recombinase polymerase amplification, polymerase chain reaction (PCR), or multiple displacement amplification. In some examples, the amplification can include digital PCR, which can run through a fixed number of thermal cycle and can do a single measurement to detect amplified DNA. This approach can utilize hundreds or thousands of individual detection chambers to build a composite detection image scaled by 1 or 0 for independent detection zones. With appropriate statistical processing digital PCR can eliminate the need for calibration curves, reference standards, or controls and can lend itself to multi-target multiplexing. The current mode circuit approach can allow for single or multiple pixel (super pixel) configurations. For example, one amplifier could be used to read individual pixels, or there could be an amplifier for individual rows or columns as in traditional CMOS imaging chips. Other applications can include detection of nucleic acids in a biological fluid.

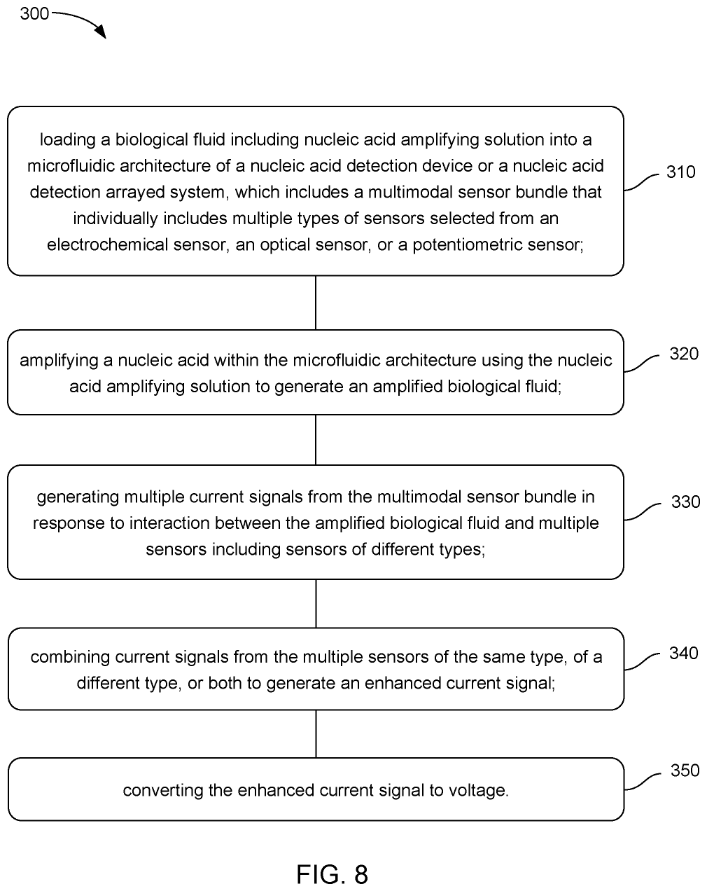

Also presented is a method of nucleic acid detection. A flow diagram of a method 300 of nucleic acid detection is shown in FIG. 8. In one example, the method includes loading 310 a biological fluid including nucleic acid amplifying solution into a microfluidic architecture of a nucleic acid detection device or a nucleic acid detection arrayed system, which includes a multimodal sensor bundle that individually includes multiple types of sensors selected from an electrochemical sensor, an optical sensor, or a potentiometric sensor. The method further includes amplifying 320 a nucleic acid within the microfluidic architecture using the nucleic acid amplifying solution to generate an amplified biological fluid, generating 330 multiple current signals from the multimodal sensor bundle in response to an interaction between the amplified biological fluid and multiple sensors including sensors of different types, combining 340 current signals from the multiple sensors of the same type, of a different type, or both to generate an enhanced current signal, and converting 350 the enhanced current signal to voltage. The nucleic acid detection device or the nucleic acid detection arrayed system can be as described above, for example. In one example, the method can include digitally enabling or disabling the electrochemical sensors, the optical sensors, the potentiometric sensors, or combination thereof using a sensor mask associated with the microfluidic architecture. In another example, the method can include gating the enhanced current signal to provide unidirectional current flow prior to converting the enhanced current signal to voltage.

In yet another example, the method can further include simultaneously measuring a real time strength of an electrochemical response generated during amplification by the redox-active intercalating dye and fluorescent emissions generated during amplification by the fluorescent intercalating dye.

As used in this specification and the appended claims, the singular forms “a,” “an,” and “the” include plural referents unless the content clearly dictates otherwise.

As used herein, a plurality of items, structural elements, compositional elements, and/or materials may be presented in a common list for convenience. However, these lists should be construed as though individual members of the list is individually identified as a separate and unique member. Thus, no individual member of such list should be construed as a de facto equivalent of any other member of the same list solely based on presentation in a common group without indications to the contrary.

Concentrations, dimensions, amounts, and other numerical data may be presented herein in a range format. A range format is used merely for convenience and brevity and should be interpreted flexibly to include the numerical values explicitly recited as the limits of the range, and also to include all the individual numerical values or sub-ranges encompassed within that range as if numerical values and sub-ranges is explicitly recited. For example, a current from 10 nA to 100,000 μA should be interpreted to include the explicitly recited limits of 10 nA to 100,000 μA, and to include currents such as about 50 nA and 1,000 μA, as well as subranges such as from 100 nA to 1,000 nA, from 500 nA to 100,000 μA, from 100 μA to 1,000 μA etc.

The terms, descriptions, and figures used herein are set forth by way of illustration and are not meant as limitations. Many variations are possible within the disclosure, which is intended to be defined by the following claims—and equivalents—in which all terms are meant in the broadest reasonable sense unless otherwise indicated.

The following illustrates an example of the present disclosure. However, the following is illustrative of the application of the principles of the present disclosure. Numerous modifications and alternative compositions, methods, and systems may be devised without departing from the scope of the present disclosure. The appended claims are intended to cover such modifications and arrangements.

### Example: Sensor Manufacture and Evaluation

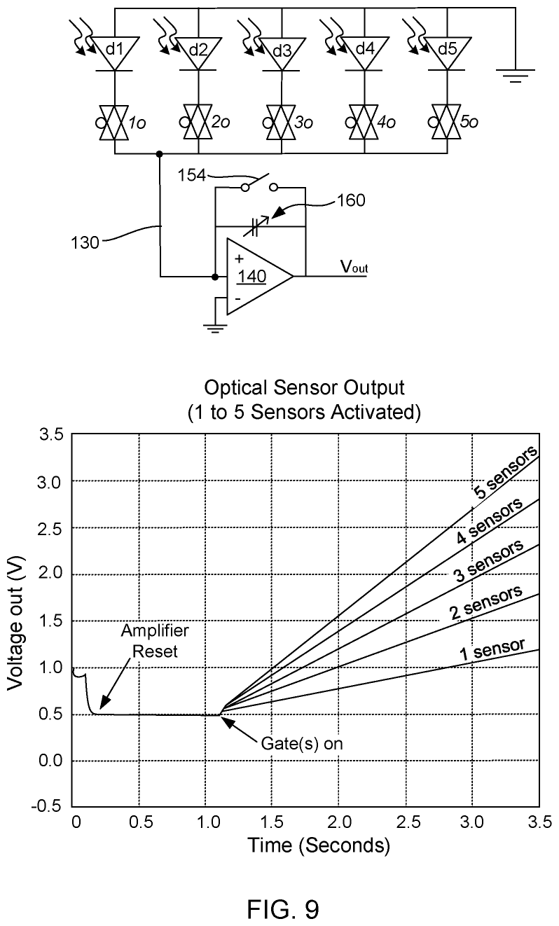

To evaluate the combining of current from multiple sensors of the same type across an array, a simple circuit was prepared as shown in FIG. 9. This simple circuit can be operated using the more complex circuitry shown in FIG. 7, for example. In FIG. 9, the circuitry included 5 optical sensor diodes d1-d5 connected in parallel through respective transmission gates 1o-5o, which feed a common transmission line 130. An amplifier or voltage converter is shown at 140 which included a reset switch 154 and feedback circuitry 160, as previously described. In this example, the five optical sensors were exposed to a biological fluid (now shown). Sensor masks or gating using the transmission gates can be used to block current from other sensors that may be present in the individual bundles. In operation of the circuit, the voltage converter was reset between readings using the reset switch. Voltage measurements were taken based on current from 1 closed transmission gate (allowing current through), 2 closed transmission gates, 3 closed transmission gates, 4 closed transmission gates, and 5 closed transmission gates. As can be seen in FIG. 9, the voltage measurements were increased with the increased number of closed transmission gates. This example evidences the ability to programmatically scale the system for increased or decreased sensitivity, which can permit the detection of nucleic acids at even low concentrations, can reduce signal-to-noise disturbances, and/or can permit a wide dynamic sensing range, for example.

The same types of measurements can be obtained by using multiple electrochemical sensors, multiple potentiometric sensor, multiple currents from a single sensor, e.g., red, green, and blue optically generated-current, multiple electrode-generated current from multiple types of electrodes, etc. Likewise, current from a common multimodal sensor bundle can be combined as well and/or can be combined with other sensor across the array.

While the present technology has been described with reference to certain examples, figures, description, and the like, various modifications, changes, omissions, and substitutions can be made without departing from the disclosure.

What is claimed is:

1. A nucleic acid detection device, comprising:
a microfluidic architecture to receive biological fluid;
a multimodal sensor bundle positioned to interact with the biological fluid when present within the microfluidic architecture, wherein the multimodal sensor bundle includes multiple types of sensors selected from an electrochemical sensor, an optical sensor, or a potentiometric sensor, wherein individual sensors of the multimodal sensor bundle independently generates a current signal in response to interaction with the biological fluid;
a common transmission line to transmit an enhanced current signal generated by combining current signals from the individual sensors; and
a current to voltage converter to receive and convert the enhanced current signal to voltage.

2. The nucleic acid detection device of claim 1, further comprising a sensor mask associated with the multimodal sensor bundle, wherein the sensor mask is electrically associated with the multimodal sensor bundle to enable or disable the individual sensors of the multimodal sensor bundle.

3. The nucleic acid detection device of claim 1, wherein the nucleic acid detection device includes multiple multimodal sensor bundles independently associated with a common transmission line to transmit an enhanced current signal generated by combining current signals from the individual sensors within the multiple multimodal sensor bundles, respectively.

4. The nucleic acid detection device of claim 1, wherein the multimodal sensor bundle includes the electrochemical sensor, the optical sensor, and the potentiometric sensor, wherein the electrochemical sensor includes a working electrode, a counter electrode, and a reference electrode, and wherein the optical sensor includes a photo-diode and a thin film filter.

5. The nucleic acid detection device of claim 1, further comprising a thermal cycler associated with the microfluidic architecture to thermally cycle the biological fluid when present within the microfluidic architecture.

6. A nucleic acid detection arrayed system, comprising:
a microfluidic architecture to receive biological fluid;
an array of multimodal sensor bundles positioned to interact with the biological fluid when present within the microfluidic architecture, wherein individual multimodal sensor bundles of the array include multiple types of sensors selected from an electrochemical sensor, an optical sensor, or a potentiometric sensor, wherein individual sensors of the multimodal sensor bundles independently generates current signal in response to interaction with the biological fluid;
a series of common transmission lines to transmit an enhanced current signal generated by combining current signals from multiple individual sensors across the array; and
a current to voltage converter to receive and convert the enhanced current signal to voltage.

7. The nucleic acid detection arrayed system of claim 6, wherein the array of multimodal sensor bundles includes the optical sensor in the form of a multi-channel optical sensor with a red channel, a green channel, and a blue channel, wherein:
current signal generated by the red channel of multiple optical sensors across the array transmit in parallel to a first common transmission line,
current signal generated by the green channel of multiple optical sensors across the array transmit in parallel to a second common transmission line, and
current signal generated by the blue channel of multiple optical sensors across the array transmit in parallel to a third common transmission line.

8. The nucleic acid detection arrayed system of claim 6, wherein the multimodal sensor bundles include the potentiometric sensor and one or both of the electrochemical sensor or the optical sensor.

9. The nucleic acid detection arrayed system of claim 6, wherein the series of common transmission lines are independently associated with a series of transmission gates, respectively, to independently gate the common transition lines prior to introduction of the enhanced current signal to the current to voltage converter.

10. The nucleic acid detection arrayed system of claim 6, wherein the current to voltage converter includes an amplifier.

11. The nucleic acid detection arrayed system of claim 6, wherein the microfluidic architecture includes microfluidic microchannels, microfluidic chambers, or both, and wherein the microfluidic architecture further includes microfluidic fluid movement network to generate fluid movement within the microfluidic architecture.

12. The nucleic acid detection arrayed system of claim 6, wherein the biological fluid is present within the microfluidic architecture and comprises a nucleic acid amplifying solution.

13. A method of nucleic acid detection, comprising:
loading a biological fluid including nucleic acid amplifying solution into a microfluidic architecture of a nucleic acid detection device or a nucleic acid detection arrayed system, which includes a multimodal sensor bundle that individually includes multiple types of sensors selected from an electrochemical sensor, an optical sensor, or a potentiometric sensor;
amplifying a nucleic acid within the microfluidic architecture using the nucleic acid amplifying solution to generate an amplified biological fluid;
generating multiple current signals from the multimodal sensor bundle in response to interaction between the amplified biological fluid and multiple sensors including sensors of different types;
combining current signals from the multiple sensors of the same type, of a different type, or both to generate an enhanced current signal; and
converting the enhanced current signal to voltage.

14. The method of nucleic acid detection of claim 13, further comprising digitally enabling or disabling the electrochemical sensor, the optical sensor, the potentiometric sensor, or combination thereof using a sensor mask associated with the microfluidic architecture.

15. The method of nucleic acid detection of claim 13, further comprising gating the enhanced current signal to provide unidirectional current flow prior to converting the enhanced current signal to voltage.
